(19) United States
(12) Patent Application Publication
(10) Pub. No.: US 2025/0022554 A1
(43) Pub. Date: Jan. 16, 2025

(54) PROGRAM, INFORMATION PROCESSING METHOD, AND INFORMATION PROCESSING DEVICE

(71) Applicant: TERUMO KABUSHIKI KAISHA, Tokyo (JP)

(72) Inventors: Satoshi SAWADA, Kanagawa (JP); Yuki SAKAGUCHI, Kanagawa (JP)

(73) Assignee: TERUMO KABUSHIKI KAISHA, Tokyo (JP)

(21) Appl. No.: 18/895,894

(22) Filed: Sep. 25, 2024

Related U.S. Application Data
(63) Continuation of application No. PCT/JP2023/008200, filed on Mar. 6, 2023.

(30) Foreign Application Priority Data
Mar. 31, 2022 (JP) 2022-061028

(51) Int. Cl. G16H 10/40

(52) U.S. Cl. CPC G16H 10/40

(57) ABSTRACT

A non-transitory computer-readable medium storing a computer program for calculating a possible administration amount of a contrast agent in consideration of the risk of developing renal damage. The program executes a process including acquiring a renal condition indicator regarding the condition of the kidney of the patient during treatment of a patient; calculating an intraoperative acceptable amount regarding a contrast agent to be administered to the patient on the basis of the renal condition indicator and patient information regarding the patient; and outputting information regarding the intraoperative acceptable amount. The patient information includes background information regarding the patient and medical history information of the patient, and the intraoperative acceptable amount is acquired by inputting the patient information, the renal condition indicator, and circulatory dynamics information of the patient into a trained model that outputs an intraoperative acceptable amount when receiving patient information, a renal condition indicator, and circulatory dynamics information.

### CROSS-REFERENCES TO RELATED APPLICATIONS

This application is a continuation of International Application No. PCT/JP2023/008200 filed on Mar. 6, 2023, which claims priority to Japanese Application No. 2022-061028 filed on Mar. 31, 2022, the entire content of both of which is incorporated herein by reference.

### TECHNOLOGICAL FIELD

The present disclosure generally relates to a program, an information processing method, and an information processing device.

### BACKGROUND DISCUSSION

A diagnostic imaging device is used when an intravascular treatment is performed. A diagnostic imaging device that sets a contrast agent injection device so as to optimize contrast of an image has been proposed (Japanese Patent Application Publication No. 2021-168814 A).

By using the diagnostic imaging device disclosed in Japanese Patent Application Publication No. 2021-168814 A, even a relatively inexperienced user can capture an image with appropriate contrast.

Meanwhile, it is known that the risk of developing renal damage increases when the dosage of a contrast agent is large. Original renal functions vary, and thus, a possible administration amount of the contrast agent without causing renal damage varies among individuals and may vary depending on various events during intravascular treatment. The diagnostic imaging device disclosed in Japanese Patent Application Publication No. 2021-168814 A cannot calculate a possible administration amount of the contrast agent in consideration of the risk of developing renal damage.

### SUMMARY

In one aspect, a non-transitory computer-readable medium storing a computer program is disclosed for calculating a possible administration amount of a contrast agent in consideration of a risk of developing renal damage.

In accordance with another aspect, a non-transitory computer-readable medium storing a computer program causes a computer to execute processing including: acquiring a renal condition indicator regarding the condition of the kidney of the patient during treatment of a patient; calculating an intraoperative acceptable amount regarding a contrast agent to be administered to the patient on the basis of the renal condition indicator and patient information regarding the patient; and outputting information regarding the intraoperative acceptable amount.

In accordance with an aspect, an information processing method executed by a computer, the method comprising: acquiring a renal condition indicator regarding a condition of a kidney of a patient during a treatment; calculating an intraoperative acceptable amount regarding a contrast agent to be administered to the patient on the basis of the renal condition indicator and patient information; and outputting information regarding the intraoperative acceptable amount.

In accordance with another aspect, an information processing device comprising a control unit, wherein the control unit is configured to: acquire a renal condition indicator regarding a condition of a kidney of a patient during a treatment; calculate an intraoperative acceptable amount regarding a contrast agent to be administered to the patient on the basis of the renal condition indicator and patient information; and output information regarding the intraoperative acceptable amount.

According to one aspect, it is possible to provide a computer program and the like for calculating a possible administration amount of a contrast agent in consideration of a risk of developing renal damage.

### BRIEF DESCRIPTION OF THE DRAWINGS

FIG. 1 is an explanatory diagram illustrating a configuration of an information processing system.

FIG. 2 is a diagram for describing a contrast agent amount model.

FIG. 3 is a flowchart for describing the flow of processing according to a program.

FIG. 4 is a screen example.

FIG. 5 is a screen example.

FIGS. 6A and 6B illustrate a screen example.

FIG. 7 is a screen example.

FIG. 8 is an example of a risk report.

FIG. 9 is a diagram for describing a renal condition model.

FIG. 10 is a flowchart for describing the flow of processing according to a program of a second embodiment.

FIG. 11 is screen example according to the second embodiment.

FIG. 12 is an explanatory diagram for describing a record layout in a treatment plan databased (DB).

FIG. 13 is an explanatory diagram for describing a plan model.

FIG. 14 is a flowchart for describing the flow of processing according to a program of a third embodiment.

FIGS. 15A and 15B illustrate an example of a graph field in the third embodiment.

FIG. 16 is an explanatory diagram for describing a configuration of an information processing system according to a fourth embodiment.

### DETAILED DESCRIPTION

Set forth below with reference to the accompanying drawings is a detailed description of embodiments of a program, an information processing method, and an information processing device.

### First Embodiment

Various types of intravascular treatments are performed such as percutaneous coronary intervention (PCI), transcatheter aortic valve implantation (TAVI), transcatheter mitral valve repair with MitraClip®, atherectomy, stent placement, and aneurysm coil embolization.

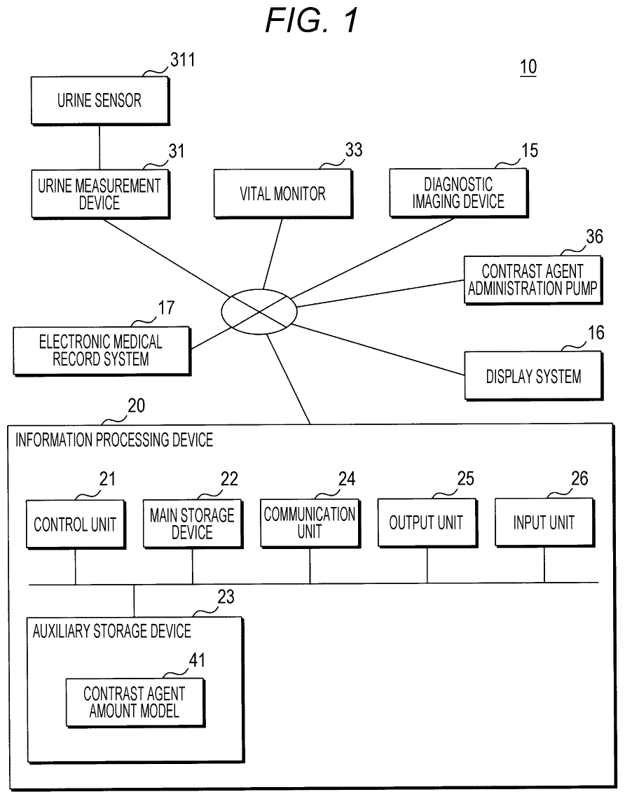

When performing these intravascular treatments, a doctor uses a diagnostic imaging device 15 (see FIG. 1) to confirm the position and state of a treatment tool inserted into the blood vessel of the patient. In the following, an example in which the diagnostic imaging device 15 captures a fluoroscopic image of a patient using X-rays will be described. When a contrast image in which a running state of a blood vessel is visualized on a fluoroscopic image is captured, an iodinated contrast agent is administered to the blood vessel of a patient in X-ray imaging or CT imaging, and a gadolinium preparation is administered to the blood vessel of a patient in a magnetic resonance imaging (MRI) or the like.

Contrast agents may cause a side effect called contrast induced-nephropathy (CIN). The higher the amount of the contrast agent administered, the higher the incidence of contrast induced-nephropathy. It is known that contrast induced-nephropathy may occur in a patient with low creatinine clearance (CCr) and estimated glomerular filtration rate (eGFR), which are indicators of renal function, even with a relatively small amount of contrast agent.

Many patients who develop contrast induced-nephropathy can be cured, for example, in several days to two weeks. However, in some patients, deterioration of renal function progresses to chronic kidney disease (CKD), and a patient with severe chronic kidney disease requires artificial dialysis. For this reason, it is desirable to administer the contrast agent in a minimum necessary amount. However, it is necessary to use a certain amount of contrast agent in order to properly perform the intravascular treatment.

Accordingly, the doctor determines an acceptable amount of the contrast agent for each patient in consideration of the balance between the risk and the benefit of the administration of contrast agent. The acceptable amount of the contrast agent can be determined on the basis of, for example, a patient background such as age, sex, and weight of the patient, information regarding the therapeutic procedure to be performed, and results of a preoperative biochemical test such as creatinine clearance and estimated glomerular filtration rate. In the following description, an acceptable amount determined before surgery may be referred to as a preoperative acceptable amount.

For example, when PCI, which is relatively simple among intravascular treatments, is performed, it is unlikely that a contrast agent in an amount exceeding an acceptable amount is required. The time required for PCI is relatively short, and thus, there is also a low possibility of a decrease in the acceptable amount of the contrast agent due to a significant change in the patient's kidney condition during surgery.

However, delicate treatments such as TAVI take a relatively long time, and thus, there is a possibility that the acceptable amount of the contrast agent will change from the preoperative acceptable amount due to a change in the patient's kidney condition during surgery. A treatment using a tool that rotates in a blood vessel, such as an atherectomy catheter and a pump catheter for auxiliary circulation, may cause renal damage due to hemolysis. Thus, there is an increased likelihood that the acceptable amount of the contrast agent will change from the preoperative acceptable amount due to a change in the patient's kidney condition during surgery. In the following description, an acceptable amount that changes during surgery may be referred to as an intraoperative acceptable amount.

When the acceptable amount of the contrast agent is greatly reduced, the doctor may, for example, alter the treatment process to reduce the number of times that the patient is subjected to contrast imaging. The doctor may use a contrast agent in an amount exceeding an acceptable amount on purpose by comprehensively determining the risk and the benefit. In order to make an accurate determination, it is desirable that the doctor can recognize a fluctuation in the acceptable amount of the contrast agent in real time.

It takes time before a serum creatinine value changes after the condition of the kidney changes. Therefore, the creatinine clearance and the estimated glomerular filtration rate used for calculating the preoperative acceptable amount change when two or three days have passed after the condition of the kidney changes. Accordingly, it is impossible to calculate the intraoperative acceptable amount in real time with the same method as the calculation of the preoperative acceptable amount. The present embodiment will describe an information processing system 10 capable of calculating an intraoperative acceptable amount in real time.

FIG. 1 is an explanatory diagram illustrating a configuration of the information processing system 10. The information processing system 10 includes an information processing device 20, a urine measurement device 31, a vital monitor 33, a diagnostic imaging device 15, a contrast agent administration pump 36, a display system 16, and an electronic medical record system 17, those of which are connected via a network such as a hospital information system (HIS).

A urine sensor 311 is connected to the urine measurement device 31. The urine measurement device 31 outputs a renal condition indicator regarding the condition of the kidney of the patient calculated using data acquired from the urine sensor 311, and the like. The renal condition indicator will be described later. The vital monitor 33 is connected to various sensors, and measures and outputs circulatory dynamics information such as blood pressure. The urine measurement device 31 and the vital monitor 33 may be integrated.

The diagnostic imaging device 15 can be, for example, an intraoperative angiography device or an intraoperative computed tomography (CT) device. The diagnostic imaging device 15 may be an intraoperative magnetic resonance imaging (MRI) apparatus. When an intraoperative MRI apparatus is used, a gadolinium preparation is often used as a contrast agent. As with the iodinated contrast agent, it is desirable to also minimize an amount of the gadolinium preparation.

The contrast agent administration pump 36 is used for administering the contrast agent to a patient. The contrast agent administration pump 36 may be directly connected to the diagnostic imaging device 15 and controlled. The contrast agent administration pump 36 may be a device that is not connected to the network or the diagnostic imaging device 15 and is manually operated by a co-medical staff such as a nurse.

The display system 16 includes a large display device that can be, for example, suspended from the ceiling of an operating room and a control system of the display system 16. The large display device can display a fluoroscopic image or a contrast image captured by the diagnostic imaging device 15, data from the vital monitor 33, and the like.

Patient information is recorded in the electronic medical record system 17. The patient information can include background information such as age, sex, height, and weight of the patient, medical history information regarding diseases the patient used to have in the past, biochemical test results, the progress of an ongoing treatment, and the like. The medical history information can include the type and amount of the contrast agent administered to the patient at the time of past treatment, the condition of the patient after administration, and the like. The progress of an ongoing treatment can include the type and amount of medicinal agent and contrast agent administered to the patient and an image captured by the diagnostic imaging device 15. Data measured by the urine measurement device 31 and the vital monitor 33 may be sequentially recorded in the electronic medical record system 17.

The information processing device 20 can include a control unit 21, a main storage device 22, an auxiliary storage device 23, a communication unit 24, an output unit 25, an input unit 26, and a bus. The control unit 21 is an arithmetic control device that executes a program according to the present embodiment. For the control unit 21, one or a plurality of central processing units (CPUs), graphics processing units (GPUs), tensor processing units (TPUs), multi-core CPUs, or the like, are used. The control unit 21 is connected to each of the hardware components constituting the information processing device 20 via the bus.

The main storage device 22 is a storage device such as a static random access memory (SRAM), a dynamic random access memory (DRAM), or a flash memory. The main storage device 22 temporarily stores information that is needed during processing performed by the control unit 21 and a program being executed by the control unit 21.

The auxiliary storage device 23 is a storage device such as an SRAM, a flash memory, a hard disk, or a magnetic tape. The auxiliary storage device 23 stores a contrast agent amount model 41, the program to be executed by the control unit 21, and various types of data necessary for executing the program. The contrast agent amount model 41 may be stored in an external mass storage device connected to the information processing device 20. The communication unit 24 is an interface that conducts communication between the information processing device 20 and the network.

The output unit 25 can be, for example, a liquid crystal display device or an organic electro-luminescence (EL) display device. The input unit 26 can be, for example, an input device such as a keyboard, a mouse, a trackball, or a microphone. The output unit 25 and the input unit 26 may be integrally stacked to constitute a touch panel.

The output unit 25 may be a connection interface that connects the information processing device 20 and an external display device. The communication unit 24 that connects data to the display system 16 or the like via a network may also serve as the output unit 25.

The information processing device 20 in the present embodiment is an information device such as a general-purpose personal computer, tablet, smartphone, or server computer. The information processing device 20 may also be a large computing machine, a virtual machine working on a large computing machine, a cloud computing system, a quantum computer, multiple computers that perform distributed processing, or the like. The information processing device 20 may be integrated with, for example, the urine measurement device 31, the vital monitor 33, the diagnostic imaging device 15, or the electronic medical record system 17.

In the following, a case where the control unit 21 mainly performs software processing will be described as an example. The processing described using flowcharts and various models may be implemented by dedicated hardware.

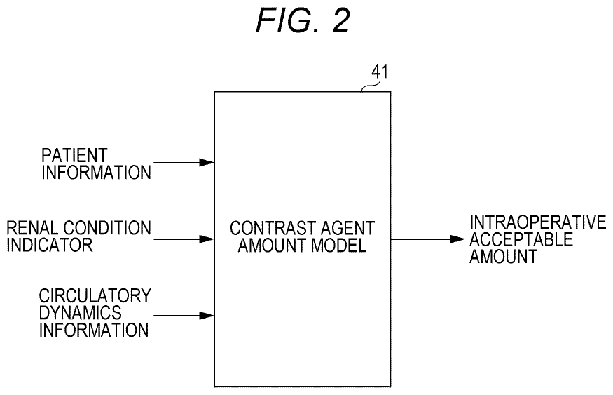

FIG. 2 is a diagram for describing the contrast agent amount model 41. The contrast agent amount model 41 is a model that receives the patient information, the renal condition indicator, and the circulatory dynamics information and outputs an acceptable amount of the contrast agent. The renal condition indicator and the circulatory dynamics information received by the contrast agent amount model 41 may be time-series data. The patient information is recorded in the electronic medical record system 17 as described above, and includes the background information, the medical history information, and the biochemical test results of the patient. The patient information may include the progress of an ongoing treatment, etc.

The renal condition indicator can be, for example, urine flow, urine volume, urine oxygen tension, urine color, absorbance of urine, amount of sodium in urine, or amount of creatinine in urine. The control unit 21 acquires the renal condition indicator from the urine measurement device 31. The control unit 21 may calculate the renal condition indicator on the basis of the measurement data acquired from the urine measurement device 31. The circulatory dynamics information can be, for example, pulse pressure, average blood pressure, heart rate, and blood oxygen saturation. The control unit 21 acquires the circulatory dynamics information from the vital monitor 33. The control unit 21 may calculate the circulatory dynamics information on the basis of the measurement data acquired from the vital monitor 33.

The urine flow can be measured in real time on the basis of, for example, a change in weight of a urine bag placed at the end of an indwelling bladder catheter. The indwelling bladder catheter may be provided with a flow sensor. Any method can be used as the principle of detecting the urine flow. The urine volume is a total amount of urine excreted from the patient's bladder after the measurement is started and can be measured in real time on the basis of the weight of the urine bag or an integrated value of urine flow.

The urine oxygen tension can be measured in real time by inserting an oxygen sensor into the bladder via the indwelling bladder catheter. The oxygen sensor may be disposed in the middle of the indwelling bladder catheter or at an inlet of the urine bag disposed at the end of the indwelling bladder catheter. Similarly, an amount of sodium in urine can be measured in real time using a sodium sensor, and an amount of creatinine in urine can be measured in real time using a creatinine sensor. Each of the oxygen sensor, the sodium sensor, and the creatinine sensor can be implemented by a sensor using a fluorescent dye or absorbance measurement.

The urine color and the absorbance of urine can be measured in real time by, for example, inserting an optical fiber connected to an optical measuring instrument such as a spectrophotometer into the indwelling bladder catheter. All of the above-mentioned renal condition indicators are described as examples. Any indicator that quickly reflects the condition of the kidney of the patient can be selected as the renal condition indicator.

The pulse pressure is a difference between the maximum blood pressure and the minimal blood pressure. The average blood pressure is a value calculated by “minimal blood pressure+(maximum blood pressure−minimal blood pressure)/3”. All of the above-mentioned circulatory dynamics information can be measured by the vital monitor 33. The circulatory dynamics information may be tissue oxygen saturation of each part of the body. Hemodynamic monitors capable of measuring tissue oxygen saturation of brain or each part of the body in real time in a minimally invasive manner are used in clinical practice.

The acceptable amount of the contrast agent output by the contrast agent amount model 41 according to the present embodiment is an intraoperative acceptable amount obtained by correcting the preoperative acceptable amount determined before surgery on the basis of the condition of the kidney of the patient. The doctor determines that the amount obtained by subtracting the amount of the contrast agent already administered to the patient from the intraoperative acceptable amount is the amount of the contrast agent that can be additionally administered to the same patient.

The contrast agent amount model 41 is a trained model trained using multiple sets of training data recorded in association with patient information, renal condition indicator, circulatory dynamics information, amount of contrast agent administered to the patient, and patient outcome. The contrast agent amount model 41 is generated using, for example, a machine learning algorithm such as XGBoost, random forest, or convolutional neural network (CNN). The contrast agent amount model 41 may be generated using an algorithm suitable for processing time-series data, such as a long short-term memory (LSTM) or a transformer.

The contrast agent amount model 41 may be a rule-based algorithm generated based on guidelines regarding the contrast agent or the like. The contrast agent amount model 41 may be a function that calculates an acceptable amount of the contrast agent using the renal condition indicator or the circulatory dynamics information as a parameter.

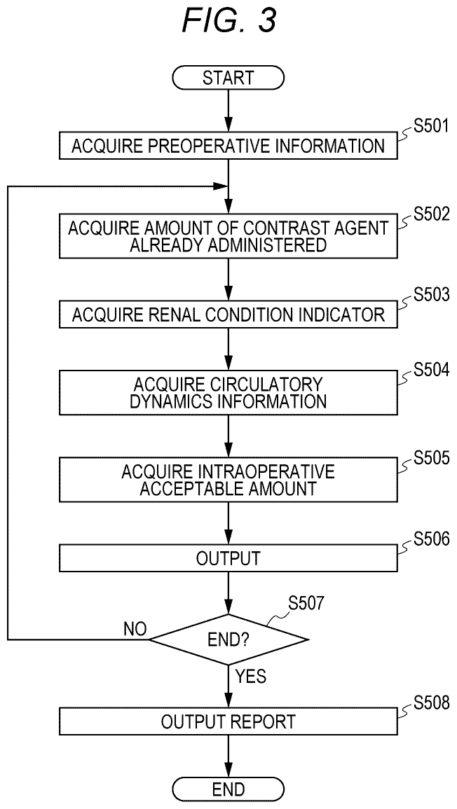

FIG. 3 is a flowchart illustrating the flow of processing according to a program. After attaching various sensors to the body of a patient, a user such as a doctor activates a program described with reference to FIG. 3.

The control unit 21 acquires patient information including background information, medical history information, and a biochemical test result of the patient, and preoperative information such as a preoperative acceptable amount of a contrast agent (step S501). The control unit 21 acquires an amount of the contrast agent already administered to the patient from the contrast agent administration pump 36 (step S502). The control unit 21 acquires the renal condition indicator from the urine measurement device 31 (step S503). The control unit 21 acquires the circulatory dynamics information from the vital monitor 33 (step S504).

The control unit 21 inputs the patient information, the renal condition indicator, and the circulatory dynamics information to the contrast agent amount model 41 to acquire an intraoperative acceptable amount (step S505). The control unit 21 outputs information regarding the intraoperative acceptable amount to the output unit 25 or the display system 16 (step S506).

The control unit 21 determines whether to end the processing (step S507). When, for example, receiving an end instruction from the user, the control unit 21 determines to end the processing. The control unit 21 may determine to end the processing when urine information and circulatory dynamics information cannot be acquired from the urine measurement device 31 and the vital monitor 33.

When determining not to end the processing (NO in step S507), the control unit 21 returns to step S502. When determining to end the processing (YES in step S507), the control unit 21 outputs a report to the output unit 25 or the display system 16 (step S508). The control unit 21 may output the report to the electronic medical record system 17. The control unit 21 ends the processing.

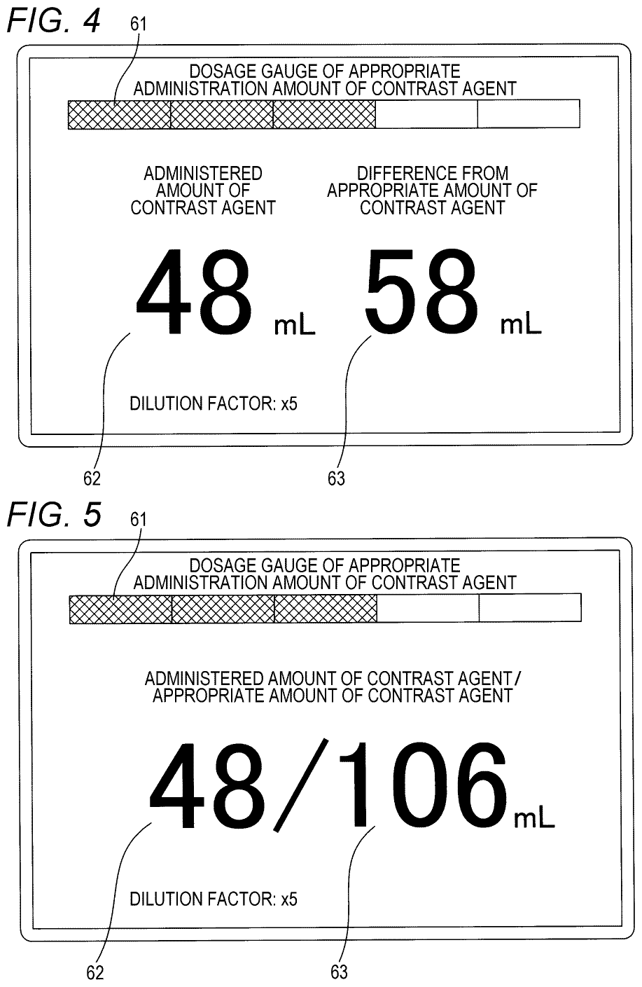

FIGS. 4 to 7 are screen examples. The control unit 21 outputs the screens illustrated in FIGS. 4 to 7 in step S506 of the program described with reference to FIG. 3. FIGS. 4 to 7 are all described as examples. The control unit 21 may receive selection regarding which screen is to be displayed from the user. The control unit 21 may receive an instruction to change items and a layout to be displayed on the screen from the user.

The screen illustrated in FIG. 4 includes a dosage gauge 61, an administered amount of contrast agent-field 62, and a difference field 63. The administered amount of contrast agent-field 62 displays the amount of the contrast agent already administered to the patient acquired in step S502 of the program described with reference to FIG. 3. The contrast agent administration pump 36 is set to dilute the contrast agent five-fold and administer the diluted contrast agent as indicated below the administered amount of contrast agent-field 62. An amount of the contrast agent before dilution is displayed in the administered amount of contrast agent-field 62.

The difference field 63 displays a difference between the intraoperative acceptable amount acquired in step S505 of the program described with reference to FIG. 3 and the contrast agent already administered to the patient. The user can recognize that the contrast agent in the amount displayed in the difference field 63 can be administered in a case where no significant change occurs in the condition of the kidney of the patient in the future. An amount of the contrast agent before dilution is also displayed in the difference field 63. The control unit 21 may receive selection of whether to display the amount of the contrast agent as the amount before dilution or the amount after dilution. The control unit 21 may receive selection to display both the amount before dilution and the amount after dilution. The user can select a display format that is determined to be preferable. The control unit 21 changes the screen on the basis of the user's selection.

The dosage gauge 61 displays the relationship between the intraoperative acceptable amount of the contrast agent and the amount of the contrast agent already administered in five stages in the lateral direction. FIG. 4 illustrates that the dosage gauge is on the third stage. The dosage gauge 61 is set to have uniform increments such as the first stage being 0% or more and less than 20%, the second stage being 20% or more and less than 40%, and the third stage being 40% or more and less than 60%.

The dosage gauge 61 may be set to have increments with arithmetic progression such as the first stage being 0% or more and less than 50%, the second stage being 50% or more and less than 75%, and the third stage being 75% or more and less than 87.5%. Alternatively, the dosage gauge 61 may be set to have any increments. The dosage gauge 61 is not limited to have five stages. The dosage gauge 61 may have four or less stages or six or more stages.

The user can intuitively recognize how much amount in the intraoperative acceptable amount has already been administered by the dosage gauge 61. Note that the intraoperative acceptable amount is the maximum amount of the contrast agent that can be administered in a state in which the risk of developing the contrast induced-nephropathy is suppressed to an appropriate range, and thus, an “appropriate administration amount of contrast agent” is displayed in the screen example.

The screen illustrated in FIG. 5 includes the dosage gauge 61, the administered amount of contrast agent-field 62, and an intraoperative acceptable amount field 64. The intraoperative acceptable amount field 64 displays the intraoperative acceptable amount acquired in step S505 of the program described with reference to FIG. 3. The administered amount of the contrast agent and the intraoperative acceptable amount are displayed in a fraction form, by which the user can easily recognize that the administered amount of the contrast agent with respect to the possible administration amount of the contrast agent is displayed.

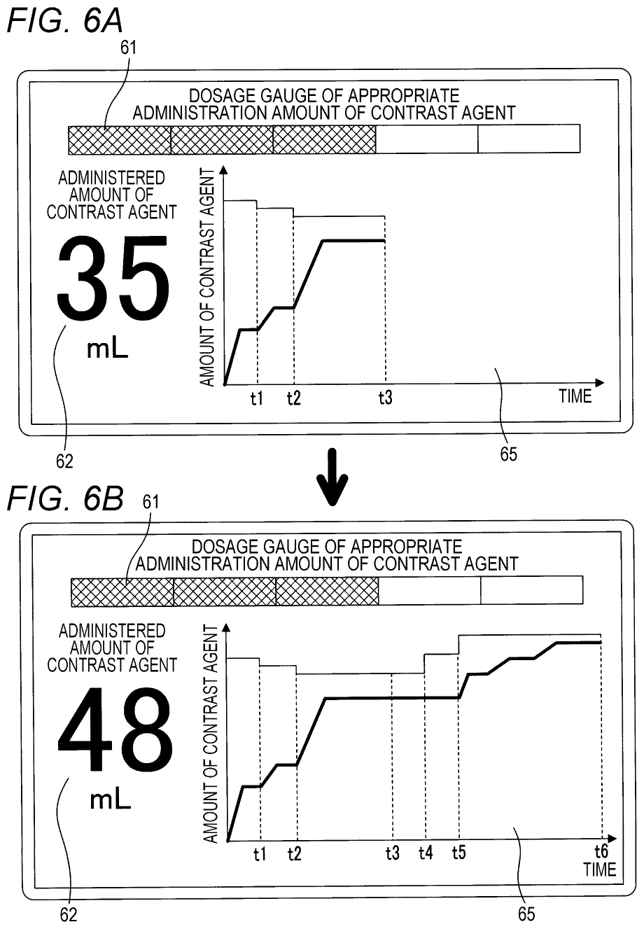

The screen illustrated in FIGS. 6A and 6B includes the dosage gauge 61, the administered amount of contrast agent-field 62, and a graph field 65. In the graph field 65, the horizontal axis represents time, and the vertical axis represents an amount of the contrast agent. A thick solid line indicates a time-series change in the administered amount of the contrast agent. A thin solid line indicates a time-series change in the intraoperative acceptable amount.

FIG. 6A illustrates a screen output from the control unit 21 at time t3. FIG. 6B illustrates a screen output from the control unit 21 at time t6. FIG. 6A will be described. The origin of the horizontal axis indicates a time point at which the program described with reference to FIG. 3 is started. The initial value of the intraoperative acceptable amount is equal to the preoperative acceptable amount determined before surgery. At time t1 and time t2, the condition of the kidney deteriorates and the intraoperative acceptable amount decreases. By time t3 which is the current time, 35 milliliters of the contrast agent has been administered.

FIG. 6B will be described. At time t4 and time t5, the condition of the kidney improves and the intraoperative acceptable amount increases. By time t6 which is the current time, 48 milliliters of the contrast agent has been administered. This dosage exceeds the intraoperative acceptable amount at time t3, but falls below the intraoperative acceptable amount at time t6.

When the intravascular treatment is still continued after time t6, the control unit 21 scrolls the graph field 65 to the left with the lapse of time so that the current time is located at the right end of the graph field 65. The user can confirm the time-series change in the intraoperative dosage and the administered amount of the contrast agent in a time range a predetermined time before the current time.

Note that the control unit 21 may gradually change the scale of the horizontal axis so that the current time is located at the right end of the graph field 65 while fixing the origin of the horizontal axis. The user can confirm the time-series change in the intraoperative dosage and the administered amount of the contrast agent in the period from the start of the intravascular treatment to the current time.

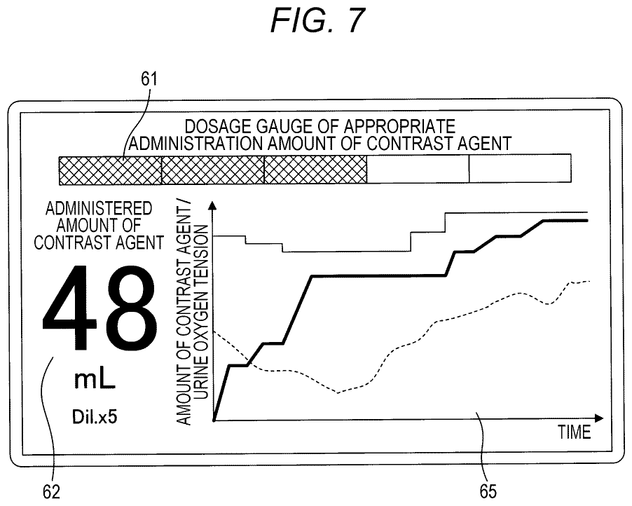

The screen illustrated in FIG. 7 displays, in the graph field 65, a time-series change in the urine oxygen tension indicated by a broken line in addition to the time-series change in the administered amount of the contrast agent and the intraoperative amount of the contrast agent. The control unit 21 may display any kind of renal condition indicator or circulatory dynamics information in the graph field 65.

As mentioned above, a doctor may use a contrast agent in an amount exceeding an intraoperative acceptable amount on purpose by comprehensively determining the risk and the benefit. When the administered amount of the contrast agent, which has temporarily exceeded the intraoperative acceptable amount, is smaller than the intraoperative acceptable amount at the end of the intravascular treatment, the risk of developing contrast induced-nephropathy is relatively low, and even if it is developed, the risk of transition to chronic kidney disease is relatively low.

There may be a case where the administered amount of the contrast agent, which is below the intraoperative acceptable amount at the time of administering the contrast agent, exceeds the final intraoperative acceptable amount at the end of the intravascular treatment due to a decrease later in the intraoperative acceptable amount caused by deterioration of the condition of the kidney. When the contrast agent is administered in an amount exceeding the final intraoperative acceptable amount, the risk of developing contrast induced-nephropathy and the risk of transition to chronic kidney disease can be relatively high. However, the risk that a patient at high risk develops contrast induced-nephropathy and the risk of transition to chronic kidney disease can be reduced by more carefully managing the condition of the patient after surgery than usual. In addition, visualizing the intraoperative acceptable amount and the administered amount of the contrast agent makes it possible to appropriately determine the process of managing the condition.

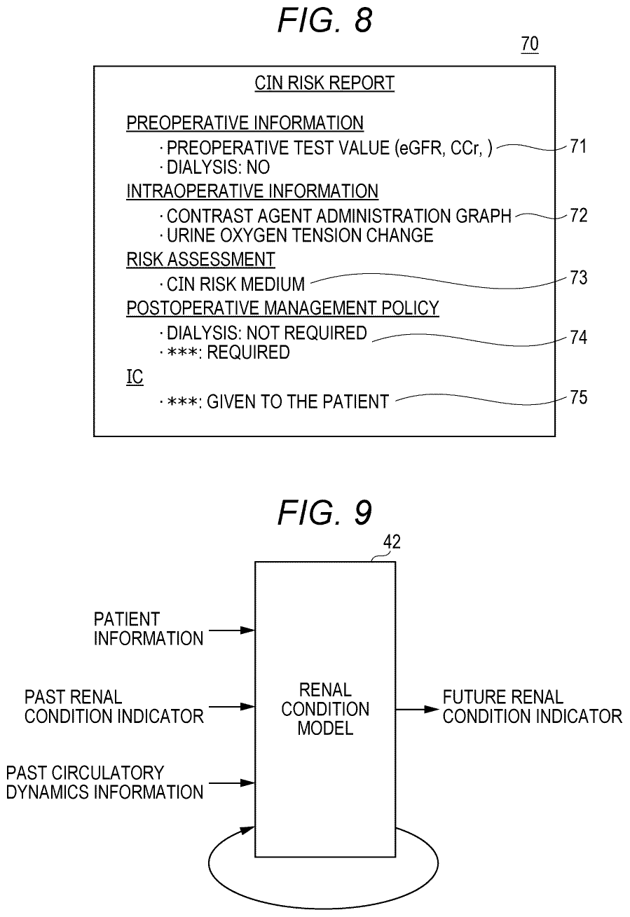

FIG. 8 is an example of a risk report 70. The control unit 21 outputs the risk report 70 illustrated in FIG. 8 in step S508 of the program described with reference to FIG. 3. The risk report 70 illustrated in FIG. 8 is an example of a report regarding the status of administration of the contrast agent output by the control unit 21 after the end of the treatment. The control unit 21 may receive an instruction to change items and a layout to be displayed in the risk report 70 from the user.

The risk report 70 can include a preoperative information field 71, an intraoperative information field 72, a risk assessment field 73, a postoperative management policy field 74, and an informed consent (IC) field 75. A part of characters in each item is a so-called hyperlink. In the following description, a case where the user browses the risk report 70 using an information device such as a personal computer or a tablet will be described as an example.

When receiving the selection of “preoperative test value (eGFR, CCr)” in the preoperative information field 71, the control unit 21 displays a result, such as the result of a biochemical test performed before the intravascular treatment, recorded in the electronic medical record system 17. The item “dialysis: no” in the preoperative information field 71 indicates that the patient had not undergone artificial dialysis before surgery.

When receiving the selection of the “contrast agent administration graph” in the intraoperative information field 72, the control unit 21 displays a graph indicating the administration record of the contrast agent illustrated in, for example, the graph field 65 of FIG. 6B. When receiving the selection of a “urine oxygen tension change” in the intraoperative information field 72, the control unit 21 displays a graph indicating a change in the urine oxygen tension illustrated in, for example, the graph field 65 of FIG. 7, in addition to the administration record of the contrast agent.

The item “CIN risk medium” in the risk assessment field 73 indicates that the patient has a medium risk of developing contrast induced-nephropathy. The control unit 21 determines the risk of developing contrast induced-nephropathy on the basis of the administered amount of the contrast agent at the end of the intravascular treatment and the intraoperative acceptable amount. For example, the control unit 21 determines the risk of developing contrast induced-nephropathy on the basis of the magnitude relationship between a determination threshold determined based on the intraoperative acceptable amount and the administered amount of the contrast agent. The control unit 21 may determine the risk of developing contrast induced-nephropathy on the basis of the ratio between the intraoperative acceptable amount and the administered amount of the contrast agent.

A specific example is now described. The control unit 21 determines that the risk is “high” when the contrast agent in an amount 1.2 times greater than the intraoperative acceptable amount has been administered, determines that the risk is “medium” when the contrast agent in an amount 1 to 1.2 times greater than the intraoperative acceptable amount has been administered, and determines that the risk is “low” when the contrast agent in an amount equal to or less than the intraoperative acceptable amount has been administered.

The factors of 1.2 and 1 are examples of a parameter for calculating the determination threshold for determining the risk, and the parameters are not limited to factors of 1.2 and 1. The threshold may be determined based on, for example, the age of the patient, whether the patient has a pre-existing condition of a kidney disease, or the like. The control unit 21 may divide the risk into four or more levels.

Contrast induced-nephropathy is an example of a complication associated with intravascular treatment. The control unit 21 may display the risk of developing complications other than the contrast induced-nephropathy.

The item “dialysis: not required” in the postoperative management policy field 74 indicates that artificial dialysis is not required in postoperative management. The item “***: required” indicates that “***” treatment is required in postoperative management. The item “***: given to the patient” in the IC field 75 indicates that an informed consent on a matter of “***” was given to the patient.

The present embodiment can provide the information processing system 10 that displays the intraoperative acceptable amount of the contrast agent which changes with the condition of the kidney of the patient in real time. The doctor can determine whether to use the contrast agent by comprehensively determining the risk and benefit of using the contrast agent.

For example, the information processing system 10 according to the present embodiment displays the intraoperative acceptable amount in real time for a patient for which the intraoperative acceptable amount is greatly reduced compared to the preoperative acceptable amount because of deterioration of the kidney condition during an intravascular treatment. The doctor can take an appropriate measure such as reducing the number of times of contrast imaging from the original schedule to reduce an amount of contrast agent used.

The information processing system 10 according to the present embodiment also displays the intraoperative acceptable amount reflecting the condition of the kidney of the patient in real time in a case where, for example, the use amount of the contrast agent approaches the preoperative acceptable amount toward the end of the intravascular treatment. The doctor can determine whether to add a contrast agent by referring to the intraoperative acceptable amount.

According to the present embodiment, it is possible to provide the information processing system 10 that assists the user to determine whether or not the amount of the contrast agent administered during the intravascular treatment has exceeded the intraoperative acceptable amount. For a patient to whom a contrast agent exceeding the intraoperative acceptable amount is administered, the doctor can prescribe a medicinal agent for protecting the kidney or perform therapeutic intervene, for example, thereby preventing the onset of contrast induced-nephropathy and chronic kidney disease.

### Modification 1-1

The control unit 21 may display an error range of the intraoperative acceptable amount. There is a measurement error in the individual data constituting the renal condition indicator and the circulatory dynamics information. The measurement error is quantified by, for example, a variation amount of raw data with respect to data obtained by smoothing time-series data which is raw data by a moving average or the like.

The control unit 21 can calculate the upper limit value and the lower limit value of the error range also for the intraoperative acceptable amount by inputting the upper limit value and the lower limit value of the measurement error range for each of the renal condition indicator and the circulatory dynamics information to the contrast agent amount model 41 described with reference to FIG. 2, for example.

The control unit 21 outputs the intraoperative acceptable amount of the lower limit value of the error range, for example. It is possible to provide the information processing system 10 that displays the intraoperative acceptable amount having a low risk even in consideration of the influence of the measurement error. The control unit 21 may output intraoperative acceptable amounts of both the upper limit value and the lower limit value of the error range. The doctor can recognize the degree of the error range of the calculated intraoperative acceptable amount and make an appropriate determination.

A safety factor may be set for the intraoperative acceptable amount. The control unit 21 outputs a value obtained by dividing the intraoperative acceptable amount output from the contrast agent amount model 41 by the safety factor. The safety factor can be set to, for example, a value of about 1.1 or 1.2. The control unit 21 may receive setting of the safety factor from the user.

A safety constant may be used instead of the safety factor. The control unit 21 outputs a value obtained by subtracting the safety constant from the intraoperative acceptable amount output from the contrast agent amount model 41. The control unit 21 may receive setting of the safety constant from the user.

It is possible to provide the information processing system 10 that displays the intraoperative acceptable amount having a low risk even in consideration of the influence of the measurement error by using the safety factor or the safety constant.

### Modification 1-2

The control unit 21 may output a control signal to the contrast agent administration pump 36 on the basis of the intraoperative acceptable amount and the administered amount of the contrast agent. For example, when the difference between the intraoperative acceptable amount and the administered amount of the contrast agent falls below a predetermined threshold, the control unit 21 outputs a control signal for reducing the amount of the contrast agent to be administered in one contrast imaging to the contrast agent administration pump 36.

The control unit 21 may display the detail of an instruction to be given before transmitting the instruction to the contrast agent administration pump 36, and transmit a control signal to the contrast agent administration pump 36 after obtaining approval from the user. It is possible to provide the information processing system 10 that reduces the use amount of the contrast agent and prevents the administration of the contrast agent in an amount exceeding the intraoperative acceptable amount when the administered amount of the contrast agent approaches the intraoperative acceptable amount.

### Modification 1-3

The control unit 21 may receive a change in setting of the dilution factor of the contrast agent from the user. For example, when the amount of the contrast agent to be administered at one time is reduced, a wide range of blood vessels can be reliably imaged by increasing the dilution factor of the contrast agent. When receiving the instruction to change the setting of the dilution factor, the control unit 21 transmits a control signal for changing the dilution factor to the contrast agent administration pump 36.

### Modification 1-4

The dilution factor of the contrast agent may be manually set by a nurse or the like. In a case where the dilution factor is manually set, the control unit 21 receives an input of the dilution factor via the input unit 26. The control unit 21 calculates the administered amount of the contrast agent before dilution on the basis of the dilution factor and the administered amount of the contrast agent acquired from the contrast agent administration pump 36, that is, the amount of the contrast agent after dilution. The control unit 21 executes the above-described series of processing using the administered amount of the contrast agent before dilution.

### Second Embodiment

The present embodiment relates to an information processing system 10 that outputs a prediction value of a renal condition indicator related to the condition of the kidney of a patient. The description of the portions same as those of the first embodiment will be omitted.

FIG. 9 is a diagram for describing a renal condition model 42. The renal condition model 42 is a trained model that receives patient information, time-series data of a past renal condition indicator, and time-series data of past circulatory dynamics information, and outputs a prediction value regarding a future renal condition indicator as time-series data.

The renal condition model 42 is generated by machine learning using a training database in which many sets of patient information, time-series data of a renal condition indicator, and time-series data of circulatory dynamics information are recorded. An algorithm suitable for processing time-series data, such as LSTM or Transformer, is used for the renal condition model 42 according to the present embodiment.

The renal condition model 42 may output prediction values of a plurality of renal condition indicators. The renal condition model 42 may output a prediction value of circulatory dynamics information.

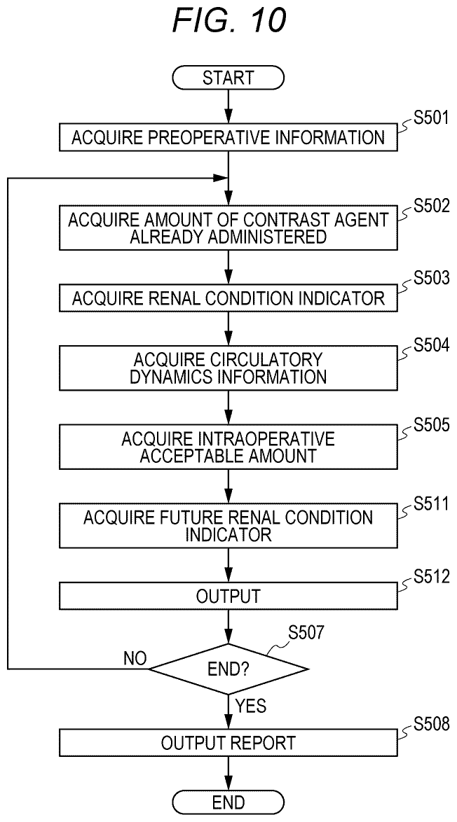

FIG. 10 is a flowchart illustrating the flow of processing according to a program of the second embodiment. The processing flow up to step S505 is the same as that according to the program of the first embodiment described with reference to FIG. 3, and therefore, the description of the processing flow up to step S505 will be omitted.

The control unit 21 inputs the patient information, the time-series renal condition indicator acquired in previous step S503, and the time-series circulatory dynamics information acquired in previous step S504 to the renal condition model 42, and acquires a prediction value of the future renal condition indicator (step S511). The control unit 21 outputs information regarding the intraoperative acceptable amount, the time-series renal condition indicator acquired in previous step S503, and the future renal condition indicator acquired in step S511 to the output unit 25 or the display system 16 (step S512).

The control unit 21 determines whether to end the processing (step S507). The subsequent processing flow is the same as the processing flow of the program according to the embodiment described with reference to FIG. 3, and thus, the description of the subsequent processing flow will be omitted. By the loop processing from step S502 to step S507, it is possible to implement the information processing system 10 that sequentially acquires the administered amount of the contrast agent, the renal condition indicator, and the circulatory dynamics information and outputs the future renal condition indicator on the basis of a temporal change of these parameters.

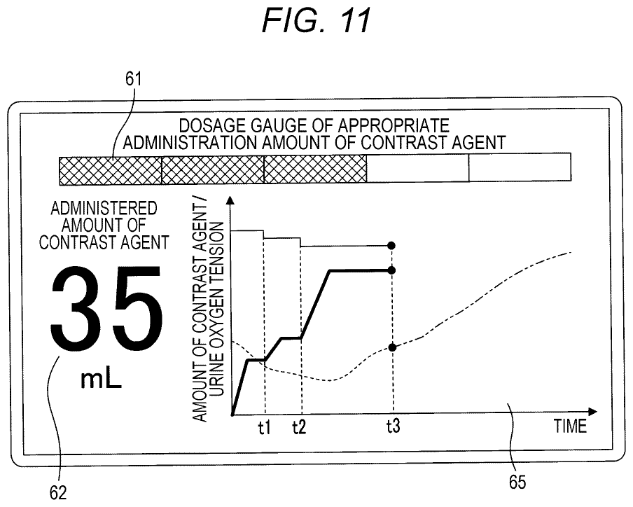

FIG. 11 is a screen example according to the second embodiment. The screen illustrated in FIG. 11 shows a state in which the urine oxygen tension, which is an example of the renal condition indicator, is added to the graph field 65 of the screen described with reference to FIGS. 6A and 6B. In the graph field 65, the horizontal axis represents time, and the vertical axis represents an amount of the contrast agent and urine oxygen tension. A thick solid line indicates a time-series change in the administered amount of the contrast agent. A thin solid line indicates a time-series change in the intraoperative acceptable amount.

FIG. 11 illustrates an example of a screen output by the control unit 21 at time t3 which is the current time. A broken line indicates a measured value of the urine oxygen tension up to time t3. A two-dot chain line indicates a prediction value of the urine oxygen tension up to time t3. Black circles indicate data at the current time.

The control unit 21 scrolls the graph field 65 to the left with the lapse of time so that the current time is located at the center of the graph field 65. The user can confirm a time-series change in the intraoperative dosage, the administered amount of the contrast agent, and the urine oxygen tension in a time range a predetermined time before the current time, and prediction of the urine oxygen tension. At this time, the manner of shift of the acceptable amount of the contrast agent after time t3 may be simultaneously predicted and displayed in the graph field 65.

The present embodiment can provide the information processing system 10 that outputs the prediction value of the renal condition indicator as time-series data. The doctor can confirm, for example, the prediction of the urine oxygen tension and take necessary measures to stabilize the patient's condition.

### Modification 2-1

Coronary Angiography (CAG) may be performed several days prior to intravascular treatment or immediately before intravascular treatment. The accuracy of the future renal condition indicator output from the renal condition model 42 described with reference to FIG. 9 can be calculated on the basis of the renal condition indicator and the circulatory dynamics information before and after the CAG is performed. The control unit 21 may display a prediction error together with the prediction value indicated by a two-dot chain line in FIG. 11 on the basis of the calculated accuracy.

### Third Embodiment

The present embodiment relates to an information processing system 10 that outputs a contrast agent use plan. The description of the portions same as those of the first embodiment will be omitted.

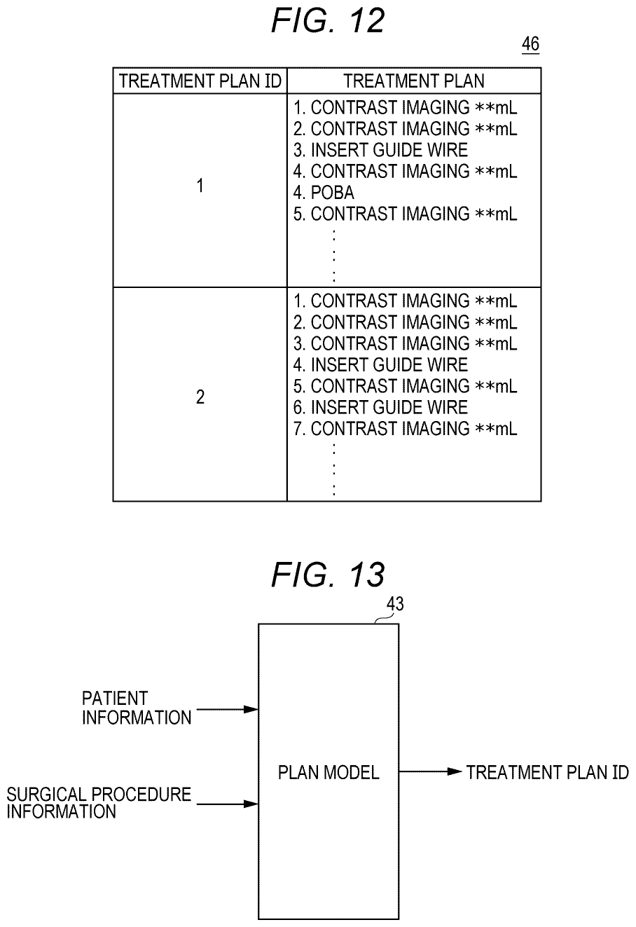

FIG. 12 is an explanatory diagram for describing a record layout in a treatment plan database (DB) 46. The treatment plan DB 46 is a database (DB) in which treatment plan numbers and treatment plans for various surgical procedures are recorded in association with each other. The treatment plan DB 46 has a treatment plan identifier (ID) field and a treatment plan field.

In the treatment plan ID field, a treatment plan ID uniquely assigned to each treatment plan is recorded. In the treatment plan field, a procedure for performing a series of treatments and an amount of the contrast agent to be administered in the procedure are recorded in chronological order. Although not illustrated, the amount of the contrast agent may be recorded as a value with a certain range such as “**±**mL”.

Each procedure recorded in the treatment plan field is a standard procedure for performing treatment such as PCI or TAVI on patients with various conditions.

For example, even when the same TAVI treatment is performed, an appropriate treatment plan varies depending on the age, body type, location of calcification, a history of an open chest surgery, general condition, and the like of the patient. For example, multiple procedures such as PCI and TAVI may be performed in a series of intravascular treatment. In the treatment plan field, treatment plans according to various situations are recorded. The treatment plan DB 46 has one record for one treatment plan.

FIG. 13 is an explanatory diagram for describing a plan model 43. The plan model 43 receives an input of patient information and surgical procedure information related to a surgical procedure scheduled for the patient and outputs a treatment plan ID. The plan model 43 is a trained model using a training database in which many sets of patient information, a surgical procedure, and a treatment plan ID selected by an experienced doctor are recorded in association with each other. The plan model 43 can be generated using, for example, a machine learning algorithm such as XGBoost, random forest, or CNN.

The plan model 43 may be a rule-based algorithm generated on the basis of a guideline defined by a medical society or the like. Instead of using the plan model 43, a doctor may select a treatment plan ID based on expertise. The plan model 43 may output a plurality of treatment plan IDs and receive selection by a doctor from among the output treatment plan IDs.

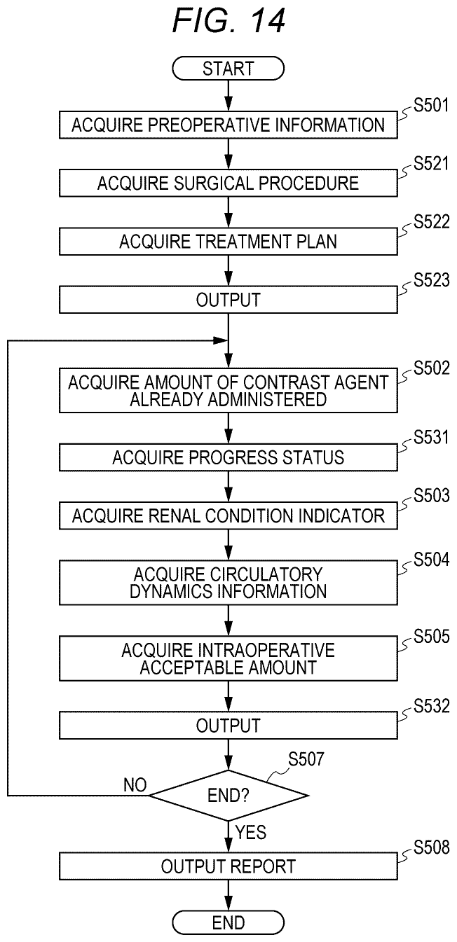

FIG. 14 is a flowchart for describing the flow of processing according to a program of the third embodiment. The control unit 21 acquires patient information including background information, medical history information, and a biochemical test result of the patient, and preoperative information such as a preoperative acceptable amount of a contrast agent (step S501). The control unit 21 acquires information regarding a surgical procedure scheduled for the patient (step S521).

The control unit 21 inputs the patient information acquired in step S501 and the surgical procedure acquired in step S521 to the plan model 43 to acquire a treatment plan ID (step S522). The control unit 21 searches the treatment plan DB 46 using the treatment plan ID acquired in step S522 as a key to extract a record. The control unit 21 outputs information regarding the treatment plan recorded in the treatment plan field of the extracted record to the output unit 25 or the display system 16 (step S523).

The control unit 21 acquires an amount of the contrast agent already administered to the patient from the contrast agent administration pump 36 (step S502). The control unit 21 acquires a treatment progress status (step S531). For example, the control unit 21 acquires a real-time image from the diagnostic imaging device 15, performs image analysis, and acquires a progress status such as insertion of a guide wire. The control unit 21 may receive input of information regarding treatment progress information from the user. The user can input the progress status by voice such as “POBA (plain old balloon angioplasty) has been completed”.

The control unit 21 acquires the renal condition indicator from the urine measurement device 31 (step S503). The control unit 21 acquires the circulatory dynamics information from the vital monitor 33 (step S504). The control unit 21 inputs the patient information, the renal condition indicator, and the circulatory dynamics information to the contrast agent amount model 41 to acquire an intraoperative acceptable amount (step S505). The control unit 21 outputs information for updating the information output in step S523 (step S532).

The control unit 21 determines whether to end the processing (step S507). When determining not to end the processing (NO in step S507), the control unit 21 returns to step S502. When determining to end the processing (YES in step S507), the control unit 21 outputs a report to the output unit 25 or the display system 16 (step S508). The control unit 21 may output the report to the electronic medical record system 17. The control unit 21 ends the processing.

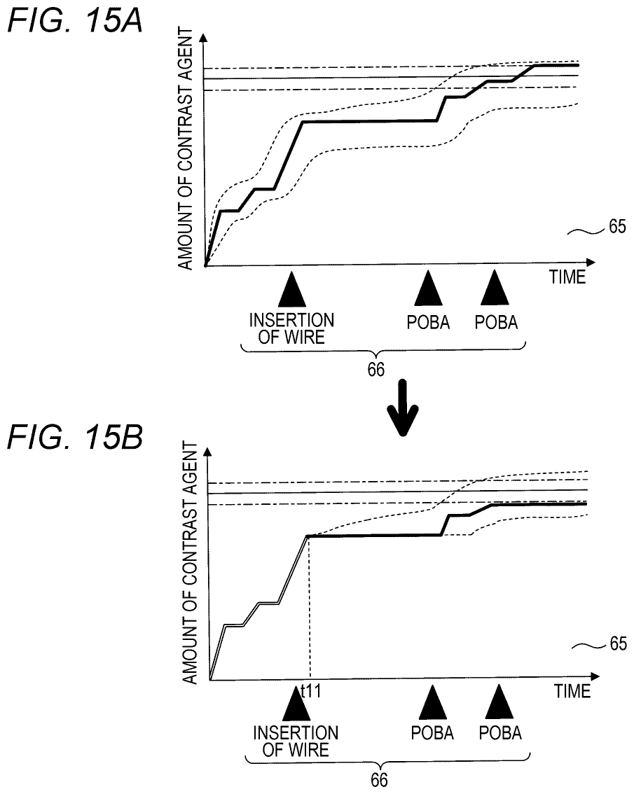

FIGS. 15A and 15B illustrate an example of a graph field 65 in the third embodiment. FIGS. 15A and 15B illustrate a region of the graph field 65 in the screen example described with reference to FIGS. 6A and 6B. The control unit 21 outputs the graph field 65 illustrated in FIG. 15A in step S523 of the program described with reference to FIG. 14. The control unit 21 outputs the graph field 65 illustrated in FIG. 15B in step S532 executed around time t11 of the program described with reference to FIG. 14.

In FIGS. 15A and 15B, a treatment progress field 66 indicating main treatment items is displayed below the horizontal axis. In the treatment plan illustrated in FIGS. 15A and 15B, POBA are performed twice after the insertion of the guide wire. A thin solid line indicates the preoperative acceptable amount. A thick solid line indicates the scheduled amount of the contrast agent to be used when the contrast agent is administered according to the treatment plan. The thick solid line in FIGS. 15A and 15B is an example of a format for outputting the contrast agent use plan.

Broken lines above and below the thick solid line indicate the upper and lower limits of the range of the contrast agent dosage included in the treatment plan. The upper limit and the lower limit may be, for example, a predetermined error range such as ±5%. Dash-dotted lines above and below the thin solid line indicate the upper limit and the lower limit of a calculation error regarding the preoperative use amount and the intraoperative use amount.

In FIG. 15A, when the contrast agent is administered along the upper limit in the treatment plan, the dosage of the contrast agent exceeds the preoperative acceptable amount between the first POBA and the second POBA. The doctor can recognize, based on the graph illustrated in FIG. 15A, that it is desirable to use the contrast agent at a dosage in a range that does not exceed a standard use amount of the contrast agent.

In FIG. 15B, the treatment plan until time t11 has been executed. The amount of the contrast agent already administered by time t11 is indicated by a double line. In FIG. 15B, the patient's kidney condition is stable, and the intraoperative acceptable amount has not varied from the preoperative acceptable amount. The contrast agent is administered according to a standard value of the treatment plan. The doctor can confirm the progress status of the treatment plan and the use amount of the contrast agent based on the graph illustrated in FIG. 15B, and can recognize that the treatment is progressing according to the treatment plan.

Note that error ranges of the preoperative dosage and the intraoperative dosage indicated by thin solid lines may be displayed in the graph field 65. An error of the amount of the contrast agent administered by the contrast agent administration pump 36 may be displayed together with the actual value indicated by a double line in FIG. 15B.

According to the present embodiment, it is possible to provide the information processing system 10 that outputs the contrast agent use plan on the basis of the treatment plan. According to the present embodiment, it is possible to provide the information processing system 10 that allows the user to rather easily confirm that the contrast agent is administered according to the treatment plan.

Note that the control unit 21 may receive modification of the treatment plan on the basis of the screen illustrated in FIG. 15A. For example, the doctor can delete the second POBA by operating the input unit 26. The control unit 21 modifies the contrast agent administration plan on the basis of the modified treatment plan and displays the modified contrast medium administration plan in the graph field 65. Specifically, the control unit 21 displays a contrast agent administration plan in which the contrast agent to be administered corresponding to the deleted POBA is reduced in the graph field 65.

### Fourth Embodiment

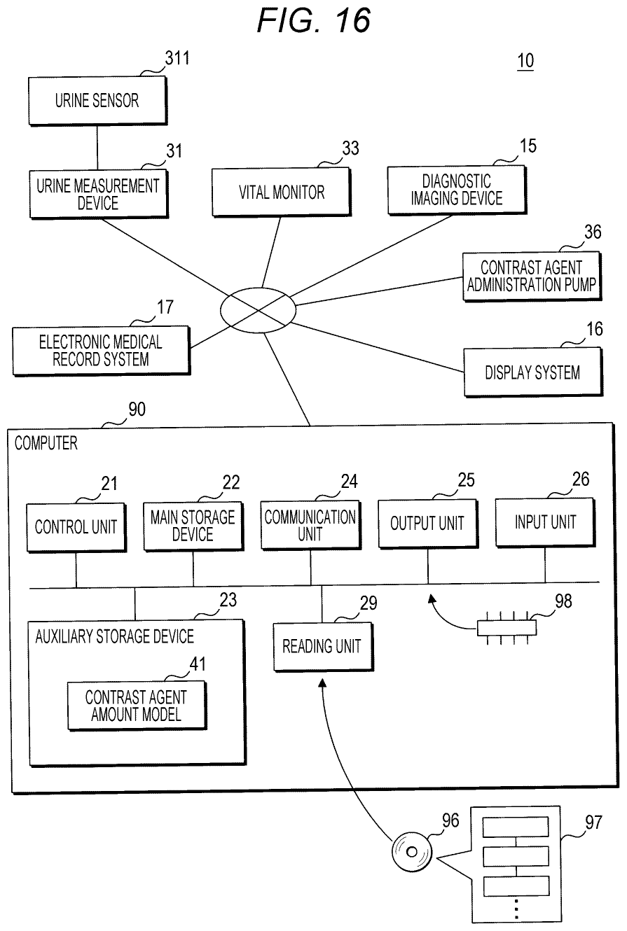

FIG. 16 is an explanatory diagram for describing a configuration of an information processing system 10 according to a fourth embodiment. The present embodiment relates to a mode that implements an information processing device 20 by running a general-purpose computer 90 and a program 97 in combination. The description of the portions same as those of the first embodiment will be omitted.

The computer 90 includes a reading unit 29 in addition to the control unit 21, the main storage device 22, the auxiliary storage device 23, the communication unit 24, the output unit 25, the input unit 26, and the bus described above.

The program 97 is recorded in a portable recording medium 96. The control unit 21 reads the program 97 via the reading unit 29 and saves the read program 97 in the auxiliary storage device 23. In addition, the control unit 21 may read the program 97 stored in a semiconductor memory 98 such as a flash memory mounted in the computer 90. Furthermore, the control unit 21 may download the program 97 from another server computer connected via the communication unit 24 and a network and save the downloaded program 97 in the auxiliary storage device 23.

The program 97 is installed as a control program for the computer 90 and is loaded into the main storage device 22 to be executed. As described above, the information processing device 20 described in the first embodiment is implemented. The program 97 in the present embodiment is an example of a program product.

The technical features (components) described in the respective embodiments can be combined with each other, and new technical features can be formed by the combination.

It should be construed that the embodiments disclosed herein are illustrative in all respects and not restrictive. The scope of the present invention is indicated not by the above meaning but by the claims, and is intended to include all changes within the meaning and scope equivalent to the claims.

(1) A non-transitory computer-readable medium storing a computer readable program that causes a computer to execute processing including: acquiring patient information regarding a patient to be treated and surgical procedure information regarding a surgical procedure scheduled for the patient; acquiring a treatment plan on the basis of the patient information and the surgical procedure information; and outputting a contrast agent use plan for the patient on the basis of the treatment plan.

(2) The non-transitory computer-readable medium storing the computer readable program according to (1), wherein the processing further includes: acquiring a preoperative acceptable amount regarding a contrast agent to be administered to the patient, the preoperative acceptable amount being calculated on the basis of the patient information; and outputting the preoperative acceptable amount along with the contrast agent use plan.

(3) The non-transitory computer-readable medium storing the computer readable program according to (1) or (2), wherein the processing further includes: receiving modification of the treatment plan; modifying the contrast agent use plan on the basis of the treatment plan that has been modified; and outputting the contrast agent use plan that has been modified.

(4) The non-transitory computer-readable medium storing the computer readable program according to any one of (1) to (3), wherein the contrast agent use plan includes information regarding an error range of a use amount of a contrast agent.

(5) The non-transitory computer-readable medium storing the computer readable program according to any one of (1) to (4), wherein the processing further includes: outputting the contrast agent use plan in a form of a graph that indicates a relationship between a progress status of a treatment plan and a scheduled amount of a contrast agent to be used; acquiring the progress status of the treatment plan; acquiring an administered amount of the contrast agent already administered to the patient; and changing the scheduled amount of the contrast agent to be used to the administered amount of the contrast agent for a region of the graph where the treatment plan has already been executed, and outputting the changed graph.

(6) The non-transitory computer-readable medium storing the computer readable program according to any one of (1) to (5), wherein the processing further includes: acquiring a renal condition indicator regarding a condition of a kidney of the patient during treatment; calculating an intraoperative acceptable amount regarding a contrast agent to be administered to the patient on the basis of the renal condition indicator and the patient information; and outputting information regarding the intraoperative acceptable amount.

(7) An information processing method executed by a computer, the method comprising: acquiring patient information regarding a patient to be treated and surgical procedure information regarding a surgical procedure scheduled for the patient; acquiring a treatment plan on the basis of the patient information and the surgical procedure information; and outputting a contrast agent use plan for the patient on the basis of the treatment plan.

(8) An information processing device comprising a control unit, wherein the control unit: acquires patient information regarding a patient to be treated and surgical procedure information regarding a surgical procedure scheduled for the patient; acquires a treatment plan on the basis of the patient information and the surgical procedure information; and outputs a contrast agent use plan for the patient on the basis of the treatment plan.

The detailed description above describes to a program, an information processing method, and an information processing device. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A non-transitory computer-readable medium storing a computer readable program that causes a computer to execute a process comprising:
acquiring a renal condition indicator regarding a condition of a kidney of a patient during a treatment;
calculating an intraoperative acceptable amount regarding a contrast agent to be administered to the patient on the basis of the renal condition indicator and patient information; and
outputting information regarding the intraoperative acceptable amount.

2. The non-transitory computer-readable medium storing the computer readable program according to claim 1, wherein the patient information includes background information regarding the patient and medical history information of the patient, and the process further comprises:
acquiring the intraoperative acceptable amount by inputting the patient information, the renal condition indicator, and circulatory dynamics information of the patient to a trained model configured to output the intraoperative acceptable amount when receiving the patient information, the renal condition indicator, and the circulatory dynamics information.

3. The non-transitory computer-readable medium storing the computer readable program according to claim 2, wherein the circulatory dynamics information includes at least one of a pulse pressure, an average blood pressure, a heart rate, blood oxygen saturation, tissue oxygen saturation, and an intravesical pressure.

4. The non-transitory computer-readable medium storing the computer readable program according to claim 1, wherein the renal condition indicator is at least one of a urine flow, a urine volume, a urine oxygen tension, and a urine color.

5. The non-transitory computer-readable medium storing the computer readable program according to claim 1, wherein the process further comprises:
sequentially acquiring the renal condition indicator;
calculating a future renal condition indicator on the basis of a temporal change in the renal condition indicator; and
outputting the calculated future renal condition indicator.

6. The non-transitory computer-readable medium storing the computer readable program according to claim 1, wherein the process further comprises:
acquiring an administered amount of the contrast agent administered to the patient; and
outputting a risk that the patient develops renal damage on the basis of the administered amount of the contrast agent and the intraoperative acceptable amount.

7. The non-transitory computer-readable medium storing the computer readable program according to claim 1, wherein the process further comprises:
acquiring an administered amount of the contrast agent administered to the patient; and
outputting a control signal to a contrast agent administration pump on the basis of a difference between the intraoperative acceptable amount and the administered amount of the contrast agent.

8. The non-transitory computer-readable medium storing the computer readable program according to claim 1, wherein the process further comprises:
outputting the renal condition indicator to a display system.

9. The non-transitory computer-readable medium storing the computer readable program according to claim 1, wherein the process further comprises:
outputting a calculation error of the intraoperative acceptable amount together with information regarding the intraoperative acceptable amount.

10. The non-transitory computer-readable medium storing the computer readable program according to claim 1, wherein the information regarding the intraoperative acceptable amount is a value obtained by setting a safety factor to the calculated intraoperative acceptable amount.

11. The non-transitory computer-readable medium storing the computer readable program according to claim 1, wherein the process further comprises:
outputting a report regarding a status of administering the contrast agent after the treatment for the patient is completed.

12. The non-transitory computer-readable medium storing the computer readable program according to claim 11, wherein the report includes the intraoperative acceptable amount, an amount of the contrast agent administered to the patient, or a risk that the patient develops a complication.

13. The non-transitory computer-readable medium storing the computer readable program according to claim 1, wherein the patient information includes information regarding the contrast agent previously administered to the patient.

14. An information processing method executed by a computer, the method comprising:
acquiring a renal condition indicator regarding a condition of a kidney of a patient during a treatment;
calculating an intraoperative acceptable amount regarding a contrast agent to be administered to the patient on the basis of the renal condition indicator and patient information; and
outputting information regarding the intraoperative acceptable amount.

15. The information processing method according to claim 14, wherein the patient information includes background information regarding the patient and medical history information of the patient, and the method further comprises:
acquiring the intraoperative acceptable amount by inputting the patient information, the renal condition indicator, and circulatory dynamics information of the patient to a trained model configured to output the intraoperative acceptable amount when receiving the patient information, the renal condition indicator, and the circulatory dynamics information.

16. The information processing method according to claim 15, wherein the circulatory dynamics information includes at least one of a pulse pressure, an average blood pressure, a heart rate, blood oxygen saturation, tissue oxygen saturation, and an intravesical pressure.

17. The information processing method according to claim 14, wherein the renal condition indicator is at least one of a urine flow, a urine volume, a urine oxygen tension, and a urine color.

18. The information processing method according to claim 14, further comprising:
sequentially acquiring the renal condition indicator;
calculating a future renal condition indicator on the basis of a temporal change in the renal condition indicator; and
outputting the calculated future renal condition indicator.

19. The information processing method according to claim 14, further comprising:
acquiring an administered amount of the contrast agent administered to the patient; and
outputting a risk that the patient develops renal damage on the basis of the administered amount of the contrast agent and the intraoperative acceptable amount.

20. An information processing device comprising a control unit, wherein the control unit is configured to:
acquire a renal condition indicator regarding a condition of a kidney of a patient during a treatment;
calculate an intraoperative acceptable amount regarding a contrast agent to be administered to the patient on the basis of the renal condition indicator and patient information; and
output information regarding the intraoperative acceptable amount.
